(19) United States
(12) Patent Application Publication
(10) Pub. No.: US 2011/0207764 A1
(43) Pub. Date: Aug. 25, 2011

(54) CYCLOPOLYSACCHARIDE COMPOSITIONS

(76) Inventors: Valery Alakhov, Ile Bizard (CA); Grzegorz Pietrzynski, Montreal (CA); Patel Kishore, Pierrefonds (CA); Thomasz Popek, Pointe-Claire (CA)

(21) Appl. No.: 13/032,159

(22) Filed: Feb. 22, 2011

Related U.S. Application Data
(60) Provisional application No. 61/338,706, filed on Feb. 23, 2010.
(60) Provisional application No. 61/399,854, filed on Jul. 19, 2010.

(51) Int. Cl. A61K 31/4745; A61K 47/40; A61K 47/42; A61P 35/00; A61P 7/02; A61P 25/04; A61P 9/00; A61P 1/08

(52) U.S. Cl. 514/283; 514/777; 514/773

(57) ABSTRACT

The present invention is directed to a composition including: (a) an active ingredient other than bendamustine; (b) a charged cyclopolysaccharide comprising at least one charged group; and (c) a stabilizing agent comprising at least one charged group having a charge opposite to that of the cyclopolysaccharide. The composition provides unexpectedly desirable stability in reactive environments such as plasma which contain entities (such as enzymes, other proteins and the like) and/or conditions which can decompose or deactivate the active ingredient.

### CROSS REFERENCE TO RELATED APPLICATION

This application claims the benefit of U.S. Provisional Patent Application Nos. 61/338,706 filed Feb. 23, 2010 and 61/399,854, filed Jul. 19, 2010, the entirety of which applications are hereby incorporated by reference into this application.

### FIELD OF THE INVENTION

The present invention is directed to a composition comprising: (a) an active ingredient other than bendamustine; (b) a charged cyclopolysaccharide comprising at least one charged group; and (c) a stabilizing agent comprising at least one charged group having a charge opposite to that of the cyclopolysaccharide.

### BACKGROUND OF THE INVENTION

Many active ingredients, particularly therapeutic agents, are introduced into environments where they encounter molecules and/or conditions which can impair their stability. For example, many pharmaceutically active ingredients have only limited solubility in aqueous environments and/or are converted into an inactive form when introduced into the bloodstream or other tissues.

The use of certain cyclopolysaccharides, in particular cyclodextrins, has been disclosed in the art. Thus, for example U.S. Pat. No. 6,583,125 discloses a composition comprising a substituted cyclodextrin and a cytotoxic compound, which composition exhibits reduced ulceration. Cyclodextrins have also been employed to increase the solubility and/or stability of a number of drugs or other materials.

However, cyclodextrins have a somewhat limited use in many of such applications, which limitations stem from the limited stability of drug-cyclodextrin complex, fast dilution of respective compositions in body fluids, and/or from the rapid clearance of cyclodextrins from circulation.

Consequently, it would be desirable to provide compositions which exhibited enhanced stability when introduced into a reactive environment.

### SUMMARY OF THE INVENTION

The present invention is directed to a composition comprising: (a) an active ingredient other than bendamustine; (b) a charged cyclopolysaccharide comprising at least one charged group; and (c) a stabilizing agent comprising at least one charged group having a charge opposite to that of the cyclopolysaccharide. Such composition provides unexpectedly desirable stability in reactive environments such as plasma which contain entities (such as enzymes, other proteins and the like) and/or conditions which can decompose or deactivate the active ingredient.

### DETAILED DESCRIPTION

The present invention is directed to a composition comprising: (a) an active ingredient; (b) a charged cyclopolysaccharide comprising at least one charged group; and (c) a stabilizing agent comprising at least one charged group having a charge opposite to that of the cyclopolysaccharide, with the proviso that the active ingredient is other than bendamustine. In one preferred embodiment, the stabilizing agent is a second charged cyclopolysaccharide having at least one charged group having a charge opposite to that of the first charged cyclopolysaccharide.

The composition provides unexpectedly desirable bioavailability and/or stability in reactive environments such as plasma which contain entities (such as enzymes, other proteins and the like) and/or conditions which can decompose or deactivate the active ingredient.

### The Active Ingredient

The active ingredient can comprise any active molecule, other than bendamustine, which has limited solubility in aqueous solutions and/or which becomes destabilized in a reactive environment. The active ingredient can be in the form of a pharmaceutically acceptable salt.

Suitable active ingredients which can be employed in the practice of this invention include: Alzheimer treatments such as donepezil; analgesics such as lamotrigine, fentanyl, lidocaine, and gabapentin; antiallergics such as cetirizine, mometasone, fexofenadine, desloratadine, fluticasone and loratadine; antiasthmatics such as montelukast, budesonide, fluticasone, and levalbuterol; antibacterials such as clarithromycin, linezolid, ciprofloxacin, azithromycin, cefdinir, and meropenem; anticholesteremic drugs such as atorvastatin, simvastatin, rosuvastatin, ezetimibe, fenofibrate, pravastatin and fluvastatin; antidepressants such as escitalopram, sertraline, duloxetine, and paroxetine; antidiabetics such as rosiglitazone and glimepiride; antiemetics such as ondansetron, terbinafine, voriconazole, and fluconazole; antihypertensives such as amlodipine, valsartan, losartan, irbesartan, metoprolol, candesartan, telmisartan, latanoprost, carvedilol, olmesartan, ramipril, nifedipine, bosentan, ramipril, enalapril, doxazosin, aand bisoprolol; antihypocalcemics such asraloxifene; anti-inflammatories such as celecoxib and meloxicam; antineoplastics such as docetaxel, anastrozole, gemcitabine, bicalutamide, tamsulosin, irinotecan, letrozole, temozolomide, erlotinib, finasteride and paclitaxel; antiobesity agents such as orlistat; antiplatelet agents such as clopidogrel; antipsychotics such as olanzapine, risperidone, quetiapine, aripiprazole and ziprasidone; antispasmodics such as tolterodine; antithyroids such as levothyroxine antivirals such as lopinavir, atazanavir and efavirenz; central nervous system stimulants such as methylphenidate, modafinil and eszopiclone; contraceptives such as drospirenone; dietary supplements such as oxcarbazepine; erectile dysfunction treatments such as sildenafil, tadalafil and vardenafil; gastrointestinal agents such as esomeprazole, lansoprazole, rabeprazole, omeprazole, tegaserod and famotidine; and immunosuppresives such as tacrolimus, cyclosporin and thalidomide.

### Cyclopolysaccharides

The cyclopolysaccharides which can be employed in the practice of this invention include cyclodextrins, cyclomannins, cycloaltrins, cyclofructans and the like. In general, cyclopolysaccharides comprising between 6 and 8 sugar units are preferred.

Among the preferred cyclopolysaccharides which can be employed are cyclodextrins.

Cyclodextrins are cyclic oligo-1-4-alpha-D-glucopiranoses comprising at least 6 sugar units. The most widely known are cyclodextrins containing six, seven or eight sugar units. Cyclodextrins containing six sugar units are known as alpha-cyclodextrins, those containing seven sugar units are known as beta-cyclodextrins and those consisting of eight sugar units are known as gamma-cyclodextrins. Particularly preferred cyclopolysaccharides are beta-cyclodextrins.

The cyclopolysaccharides employed comprise at least one charged group. The charged group can be anionic, in which case the stabilizing agent is cationic; or the group can be cationic, in which case the stabilizing agent is anioinic. Preferred anionic groups include carboxyl, sulfonyl and sulphate groups; while preferred cationic groups include amino, guanidino, and quarternary ammonium groups.

As is employed herein the term “charged cyclopolysaccharide” refers to a cyclopolysaccharide having one or more of its hydroxyl groups substituted or replaced with a charged group. The term “charged” is intended to include groups or moieties which become charged under the conditions in which the compositions of the invention are manufactured. Such moiety can itself be a charged or chargeable group (e.g., such as a sulfonyl group) or it can comprise an organic moiety (e.g., a C₁-C₆ alkyl or C₁-C₆ alkyl ether moiety) substituted with one or more charged groups. The number of substituting groups per one molecule of cyclopolysacharide can vary from 1 to the total number of hydroxyl groups in the molecule, which depends on the structure of cyclopolysacharide, and for example in beta-cyclodextrin it is 21, which is three groups per each of seven sugar residues in beta-cyclodextrin. It is preferred that average number of substitution is at least 0.5 per sugar residue, and particularly preferred is that it is about 1 per sugar residue, which for example is on average 7 (between 6 and 8) per molecule of beta-cyclodextrin.

When an anionic cyclopolysaccharide is employed, the compound can comprise any one or mixture of anionic groups. It is preferred that the anionic cyclopolysaccharide compound comprises a carboxyl, sulfonyl, or sulphate group. Preferred anionic cyclopolysaccharides include sulfobutyl ether beta-cyclodextrin, carboxymethylated-beta-cyclodextrin, O-phosphated-beta-cyclodextrin, succinyl-(2-hydroxy)propyl-beta-cyclodextrin, sulfopropylated-beta-cyclodextrin, and O-sulfated-beta-cyclodextrin with sulfobutyl ether beta-cyclodextrin being particularly preferred.

When a cationic cyclopolysaccharide is employed, such compound can comprise any one or mixture of cationic groups. It is preferred that the cationic cyclopolysaccharide comprises an amino, a guanidine or a quarternary ammonium group. Suitable amino-cyclodextrins which can be employed are amino-alpha-cyclodextrins, amino-beta-cyclodextrins, and amino-gamma-cyclodextrins, preferably having a substitution level of between about 4 and about 10. Preferred amino-cyclodextrins of this type include hexakis(6-amino-6-deoxy)alpha-cyclodextrin, heptakis(6-amino-6-deoxy)beta-cyclodextrin, and octakis(6-amino-6-deoxy)gamma-cyclodextrin. Other cationic cyclopolysaccharides which can be employed include guanidino-cyclodextrins, preferably having a substitution level of between about 4 and about 10, such as heptakis(6-guanidino-6-deoxy)beta-cyclodextrin; alkylamino-cyclodextrins, preferably having a substitution level of between about 4 and about 10, such as 6-deoxy-6-(3-hydroxy)propylamino beta-cyclodextrin; and ammonium-cyclodextrins, preferably having a substitution level between 4 and 9, such as 2-hydroxy-N,N,N-trimethylpropanammonium-cyclodextrin.

Particularly preferred cationic polysaccharides include hexakis(6-amino-6-deoxy)alpha-cyclodextrin, heptakis(6-amino-6-deoxy)beta-cyclodextrin, octakis(6-amino-6-deoxy)gamma-cyclodextrin, heptakis(6-guanidino-6-deoxy)beta-cyclodextrin, octakis(6-guanidino-6-deoxy)-gamma-cyclodextrin, 2-hydroxy-N,N,N-trimethylpropanammonium-cyclodextrin and 6-deoxy-6-(3-hydroxy)propylamino beta-cyclodextrin.

### Cationic Stabilizing Agents

In those embodiments wherein the cyclopolysaccharide is modified with anionic groups, the stabilizing agent is selected from cationic agents, or from polycationic compounds. Cationic agents which can be employed include primary amines, secondary amines, tertiary amines or quaternary ammonium compounds, such as N-alkyl-N,N-dimethylamines, N-alkyl-N,N-diethylamines, N-alkyl-N-N-diethanoloamines, triethanoloamine, N-alkylmorpholine, N-alkylpiperidine, N-alkylpyrrolidine, N-alkyl-N,N,N-trimethylammonium, N,N-dialkyl-N,N-dimethylammonium, N-alkyl-N-benzyl-N,N-diimethylammonium, N-alkyl-pyridinium, N-alkyl-pico linium, alkylamidomethylpyridinium, carbalkoxypyridinium, N-alkylquino linium, N-alkylisoquino linium, N,N-alkylmethylpyrollidinium, and 1-alkyl-2,3-dimethylimidazolium. Particularly preferred cationic adjuvants include sterically hindered tertiary amines, such as N-alkyl-N-N-diisopropylamine, N-alkylmorpholine, N-alkylpiperidine, and N-alkylpyrrolidine; and quaternary ammonium compounds such as cetylpyridinium chloride, benzyldimethyldodecylammonium chloride, dodecylpyridinium chloride, hexadecyltrimethylammonium chloride, benzyldimethyltetradecylammonium chloride, octedecyldimethylbenzylammonium chloride, and domiphen bromide.

Polycationic compounds such as oligo- or polyamines, or pegylated oligo- or polyamines can also be employed as the stabilizing agent. Preferred polycationic compounds include oligoamines such as spermin, spermidin, putrescine, and cadaverine; polyamines: such as polyethyleneimine, polyspermin, polyputrescine, and polycadaverine; and pegylated oligoamines and polyamines of the group listed above. Particularly preferred is PI2080, polyethyleneimine 2000 conjugated with PEG 8000.

One preferred class of cationic stabilizing agents are polypeptides comprising from about 5 to about 50, more preferably between about 6 and about 20, amino acids; wherein at least about 50% of such amino acids contain a positive charge. Most preferably, such charged amino acid is arginine. Particularly preferred members of this class of peptides include arginine rich peptides comprising at least one block sequence of 4 arginines. Another particularly preferred member of this class of peptides is protamine which has been digested with thermolysin (hereinafter referred to as Low Molecular Weight Protamine or “LMWP”).

Hydrophobically modified oligo- or polyamines can also be employed. Preferred stabilizing agents of this type include acetyl spermin, acetyl polyspermin, acetyl polyethyleneimine, butyryl spermin, butyryl polyspermin, butyryl polyethyleneimine, lauroyl spermin, lauroyl polyspermin, lauroyl polyethyleneimine, stearoyl spermin, stearoyl polyspermin, and stearoyl polyethyleneimine.

In addition, cationic polysaccharides and synthetic polycationic polymers can also be employed. Suitable cationic polysaccharides are chitosan, deacetylated chitosan, quaternized cellulose, quaternized amylose, quaternized amylopectine, quaternized partially hydrolyzed cellulose, quaternized partially hydrolyzed amylose and quaternized partially hydrolyzed amylopectine. Suitable synthetic polycationic polymers are Polyquaternium 2 (poly[bis(2-chloroethyl]ether-alt-1,3-bis[3-dimethylamino)propyl]-urea quaternized); Polyquaternium 11 (poly(1-vinylpyrrolidone-co-dimethylammonioethyl methacrylate) quaternized); Polyquaternium 16 and 44 (copolymer of vinylpyrrolidone and quaternized vinylimidazole); and Polyquaternium 46 (copolymer of vinylcaprolactam, vinylpyrrolidone and quaternized vinylimidazole).

One particularly preferred class of cationic stabilizing agents are cationic cyclopolysaccharide compounds, particularly cationic cyclodextrins. When such a cationic cyclopolysaccharide is employed as the stabilization agent, such compound can comprise any one or mixture of cationic groups. It is preferred that such compound comprises an amino, a guanidine or a quarternary ammonium group. Suitable amino-cyclodextrins which can be employed are amino-alpha-cyclodextrins, amino-beta-cyclodextrins, and amino-gamma-cyclodextrins, preferably having a substitution level of between about 4 and about 10. Preferred amino-cyclodextrins of this type include hexakis(6-amino-6-deoxy)alpha-cyclodextrin, heptakis(6-amino-6-deoxy)beta-cyclodextrin, and octakis(6-amino-6-deoxy)gamma-cyclodextrin. Other cationic cyclopolysaccharides which can be employed include guanidino-cyclodextrins, preferably having a substitution level of between about 4 and about 10, such as heptakis(6-guanidino-6-deoxy) beta-cyclodextrin; alkylamino-cyclodextrins, preferably having a substitution level of between about 4 and about 10, such as 6-deoxy-6-(3-hydroxy)propylamino beta-cyclodextrin; and ammonium-cyclodextrins, preferably having a substitution level between 4 and 9, such as 2-hydroxy-N,N,N-trimethylpropanammonium-cyclodextrin.

Particularly preferred cationic polysaccharides include hexakis(6-amino-6-deoxy)alpha-cyclodextrin, heptakis(6-amino-6-deoxy)beta-cyclodextrin, octakis(6-amino-6-deoxy)gamma-cyclodextrin, heptakis(6-guanidino-6-deoxy)beta-cyclodextrin, octakis(6-guanidino-6-deoxy)-gamma-cyclo dextrin, 2-hydroxy-N,N,N-trimethylpropanammonium-cyclodextrin and 6-deoxy-6-(3-hydroxy)propylamino beta-cyclodextrin.

### Anionic Stabilizing Agents

In those embodiments wherein the cyclopolysaccharide is modified with cationic groups, the stabilizing agent is selected from anionic agents, or from polyanionic polymers.

Preferably, such anionic agent is selected from compounds comprising a carboxy-, sulfate-, sulfono-, phosphate-, or phosphono-group.

One class of anionic agents that can be employed are anionic surfactants such as sodium 3-[(3-cholamidopropyl)dimethylammonio]-1-propanesulfonate, sodium N-lauroylsarcosinate, sodium dodecyl sulfate, sodium dodecylbenzylsulfonate and the like.

Anionic polysaccharides can also be employed as the stabilizing agent. Suitable compounds are chondroitin sulfate, dermatan sulphate, kappa-carrageenan, iota-carrageenan, lambda-carrageenan, mu-carrageenan, xi-carrageenan, psi-carrageenan, tau-carrageenan, furcellaran, heparan sulphate, keratin, fucoidan, hyaluronic acid, alginic acid, poly(sulfonylbutylo)cellulose, poly(sulfonylpropylo)cellulose, poly(sulfonylpropylo)dextran, poly(sulfonylbutylo)dextran, poly(sulfonylbutylo)amylase and poly(sulfonylpropylo)amylase.

The stabilizing agent can also be a polyanionic polymer selected from polyacrylates, polymethacrylates, and their copolymers.

One preferred class of anionic stabilizing agents cyclopolysaccharide compounds, particularly anionic cyclodextrins. When an anionic cyclopolysaccharide is employed as the stabilizing agent, such compound can comprise any one or mixture of anionic groups. However, in general, it is preferred that such compound comprises a carboxyl, sulfonyl, or sulphate group. Preferred anionic cyclopolysaccharides include sulfobutyl ether beta-cyclodextrin, carboxymethylated-beta-cyclodextrin, O-phosphated-beta-cyclodextrin, succinyl-(2-hydroxy)propyl-beta-cyclodextrin, sulfopropylated-beta-cyclodextrin, and O-sulfated-beta-cyclodextrin with sulfobutyl ether beta-cyclodextrin being particularly preferred.

In one particularly preferred embodiment of this invention, the first charged cyclopolysaccharide comprises sulfobutyl ether beta-cyclodextrin and the stabilizing agent comprises 6-deoxy-6-(3-hydroxy)propylamino beta-cyclodextrin.

### Excipients

The compositions of this invention can further contain pharmaceutically acceptable excipients, such as sugars, polyalcohols, soluble polymers, salts and lipids.

Sugars and polyalcohols which can be employed include, without limitation, lactose, sucrose, mannitol, and sorbitol.

Illustrative of the soluble polymers which can be employed are polyoxyethylene, poloxamers, polyvinylpyrrolidone, and dextran.

Useful salts include, without limitation, sodium chloride, magnesium chloride, and calcium chloride.

Lipids which can be employed include, without limitation, fatty acids esters, glycolipids, and phospholipids.

### Preparation

Preferably, the proportion of active ingredient to charged cyclopolysaccharide, by weight, is between about 1:12,500 and about 1:5; is more preferably between about 1:5,000 and about 1:10; and most preferably between about 1:1,500 and 1:10.

The composition of the invention can be prepared by the dissolution of the active ingredient in an aqueous solution of the cyclopolysaccharide; or by mixing an aqueous solution of the cyclopolysaccharide with an aqueous stock solution of the active ingredient. Such resulting mixture is mixed and optionally subjected to the action of ultrasound waves and/or heat to obtain an homogenous and equilibrated aqueous solution. When the cyclopolysaccharide is a cyclodextrin, it is preferred that the aqueous solution of cyclodextrin used for the preparation of composition contains at least 4% of cyclodextrin; more preferably such solution contains at least 10% of cyclodextrin.

The stabilizing agent and excipient (if present) are preferably introduced to the composition by their addition to a pre-prepared aqueous homogenous and equilibrated solution of the active ingredient with cyclopolysaccharide. Such agents can be added either as pure substances or as aqueous solutions and are preferably mixed employing agitation. Preferably, the final composition is filtered before use for injection.

The composition can be optionally freeze-dried to produce a solid material suitable for dissolution in injection media before its use. It is preferred that compositions comprising amines as stabilizing agents are freeze dried prior to the addition of such stabilizing agent, with such agent being introduced into the composition after reconstitution, shortly before use.

In one embodiment the composition of this invention is prepared by mixing the components and incubation.

In another embodiment the composition of this invention is prepared by mixing the components and applying ultrasound to the mixture.

In another embodiment the composition of this invention is prepared by mixing the components, incubation, and freeze-drying the product.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### EXAMPLES

### Example 1

### Preparation of a Composition Comprising Coumarin, Sulfobutyl Ether Beta-cyclodextrin (“SBECD”), and Cetylpyridinium Chloride

200 mg of coumarin were added to 4 g of 60% (w/w) aqueous SBECD, and the mixture was mixed at 34° C. for 48 hours, and then filtered through a 0.2 micrometer nylon filter to produce 44.3 mg/g coumarin solution, as determined by HPLC. 40 mg of cetylpiridinium chloride were added to the solution and mixed until completely dissolved, and the composition was filtered.

### Example 2

### Preparation of a Composition Comprising Ibuprofen, Sulfobutyl Ether Beta-cyclodextrin, and Triethanoloamine

400 mg of ibuprofen were added to 4 g of 60% (w/w) aqueous SBECD, and the mixture was mixed at 34° C. for 48 hours, and then filtered through a 0.2 micrometer nylon filter to produce 78.1 mg/g ibuprofen solution, as determined by HPLC. 60 mg of triethanoloamine were added to the solution and mixed until completely dissolved, and the composition was filtered.

### Example 3

### Preparation of a Composition Comprising Paclitaxel, Sulfobutyl Ether Beta-cyclodextrin, and Heptakis(6-guanidino-6-deoxy)-beta-cyclodextrin

2 mg of paclitaxel were added to 4 g of 60% (w/w) aqueous SBECD, and the mixture was mixed at 34° C. for 48 hours, and then filtered through a 0.2 micrometer nylon filter to produce 0.4 mg/g paclitaxel solution, as determined by HPLC. 40 mg of heptakis(6-guanidino-6-deoxy)-beta-cyclodextrin were added to the solution and mixed until completely dissolved, and the composition was filtered.

### Example 4

### Preparation of a Composition Comprising Diindolylmethane, Sulfobutyl Ether Beta-cyclodextrin, and Octakis(6-guanidino-6-deoxy)-gamma-cyclodextrin

200 mg of diindolylmethane were added to 4 g of 60% (w/w) aqueous SBECD, and the mixture was heated in boiling water bath for 1 hour, then kept at room temperature for 24 hours. The mixture was then filtered through a 0.2 micrometer nylon filter to produce 40.3 mg/g diindolylmethane solution, as determined by HPLC. 24 mg of octakis(6-guanidino-6-deoxy)-gamma-cyclodextrin were added to the solution and mixed until completely dissolved, and the composition was filtered.

### Example 5

### Preparation of a Composition Comprising Semaxanib, Sulfobutyl Ether Beta-cyclodextrin, and PI2080

10 mg of Semaxanib (3-((3,5-dimethyl-1H-pyrrol-2-yl)methylene)indolin-2-one) were added to 4 g of 60% (w/w) aqueous SBECD, and the mixture was heated in boiling water bath for 1 hour, then kept at room temperature for 24 hours. The mixture was then filtered through a 0.2 micrometer nylon filter to produce 1.3 mg/g semaxanib solution, as determined by HPLC. 80 mg of PI2080 (polyethyleneimine 2000 conjugated with PEG 8000) were added and mixed until completely dissolved, and the composition was filtered.

### Example 6

### Preparation of a Composition Comprising Xanthone, Sulfobutyl Ether Beta-cyclodextrin, and Low Molecular Weight Protamine

40 mg of xanthone (9H-xanthen-9-one) were added to 4 g of 60% (w/w) aqueous SBECD, and the mixture was heated in boiling water bath for 1 hour, then kept at room temperature for 24 hours. The mixture was filtered through a 0.2 micrometer nylon filter to produce 8.2 mg/g xanthone solution, as determined by HPLC. 120 mg of low molecular weight protamine were added and mixed until completely dissolved, and the composition was filtered.

### Example 7

### Preparation of a Composition Comprising Carvedilol, Sulfobutyl Ether Beta-cyclodextrin, and Hexakis(6-amino-6-deoxy)-alpha-cyclodextrin

100 mg of carvedilol were added to 4 g of 60% (w/w) aqueous SBECD, and the mixture was heated in boiling water bath for 1 hour, then kept at room temperature for 24 hours. The mixture was filtered through a 0.2 micrometer nylon filter to produce 22.6 mg/g carvedilol solution, as determined by HPLC. 120 mg of hexakis(6-amino-6-deoxy)-alpha-cyclodextrin were dissolved in 2 mL water, and the two solutions mixed until completely homogenous. The product solution was freeze-dried to form an amorphous solid.

### Example 8

### Preparation of a Composition Comprising Talidomid, Sulfobutyl Ether Beta-cyclodextrin, and Heptakis(6-amino-6-deoxy)-beta-cyclodextrin

10 mg of talidomid were added to 4 g of 60% (w/w) aqueous SBECD, and the mixture was mixed at 34° C. for 48 hours. The mixture was filtered through a 0.2 micrometer nylon filter to produce 1.7 mg/g talidomid solution, as determined by HPLC. 120 mg of heptakis(6-amino-6-deoxy)-beta-cyclodextrin were added and mixed until completely dissolved, and the composition was filtered.

### Example 9

### Preparation of a Composition Comprising SN-38, Sulfobutyl Ether Beta-cyclodextrin, and Heptakis(6-amino-6-deoxy)-beta-cyclodextrin

2 mg of SN-38 (7-Ethyl-10-hydroxy-camptothecin) were added to 4 g of 60% (w/w) aqueous SBECD, and the mixture was heated in boiling water bath for 1 hour, then kept at room temperature for 24 hours. The mixture was filtered through a 0.2 micrometer nylon filter to produce 0.39 mg/g SN-38 solution, as determined by HPLC. A solution of 60 mg of heptakis(6-amino-6-deoxy)-beta-cyclodextrin in 2 mL water was added to the SN-38 solution, and mixed until the resulting solution was completely homogenous. The composition was filtered.

### Example 10

### Preparation of a Composition Comprising Megesterol, Sulfobutyl Ether Beta-cyclodextrin, and Octakis(6-amino-6-deoxy)-gamma-cyclodextrin

20 mg of megesterol were added to 4 g of 60% (w/w) aqueous SBECD, and the mixture was heated in boiling water bath for 1 hour, then kept at room temperature for 24 hours. The mixture was filtered through a 0.2 micrometer nylon filter to produce 4.3 mg/g megesterol solution, as determined by HPLC. 60 mg of octakis(6-amino-6-deoxy)-gamma-cyclodextrin were added and mixed until completely dissolved. The product was dried in vacuum to produce an amorphous powder.

### Example 11

Pharmacokinetics of SN-38 and SN-38 glucoronide (SN-38G) upon dosing of composition comprising SN-38, SBECD, and low molecular weight protamine (LMWP) intravenously to rats.

A group of 4 female Sprague-Dawley rats received i.v. injections of SN-38 (2 mg/kg) in 40% SBECD with 1% LMWP. Blood plasma samples were collected from each animal, extracted using liquid extraction with cold methanol/acetonitrile mixture (1:1 v/v), and analyzed using a HPLC method with fluorescent detection. The results are presented in Table 1 below.

TABLE 1
Plasma levels of SN38 and SN-38G upon dosing of a
composition comprising SN-38 (dose 2 mg/kg), 40% SBECD,
and 1% low molecular weight protamine (LMWP)

| Time | SN-38 | SN-38G |
|---|---|---|
| [h] | [ng/mL] | [ng/mL] |
| 0.17 | 933.0 | 1960.9 |
| 0.5 | 343.6 | 1242.0 |
| 0.75 | 237.3 | 1049.0 |
| 1 | 122.6 | 677.4 |
| 3 | 10.4 | 115.8 |
| 6 | 7.2 | 38.6 |
| 10 | 6.2 | 24.9 |
| 24 | — | 4.5 |

Calculated values of area under the curve (AUC) for SN-38 and SN-38G:

| AUC of SN38 | 556 ng*h/mL |
|---|---|
| AUC of SN-38G | 2362 ng*h/mL |
| Ratio AUC SN-38/SN-38G | 0.24 |

This result illustrates that the composition of the present invention provides the pharmaceutical agent (SN-38) with systemic bioavailability as demonstrated by the formation of the main metabolite of the compound, SN-38G.

### Example 12

Pharmacokinetics of SN-38 and SN-38 glucuronide (SN-38G) upon dosing of a composition comprising SN-38, SBECD, and heptakis(6-amino-6-deoxy)-beta-cyclodextrin (H6A) intravenously to rats.

Female Sprague-Dawley rats, 4 animals per group received i.v. injections of SN-38 (0.65 mg/kg or 2 mg/kg) in 40% SBECD with either 1% H6A or 2% H6A. Blood plasma samples were collected from each group of animals, extracted using liquid extraction with cold methanol/acetonitrile mixture (1:1 v/v), and analyzed using a HPLC method with fluorescent detection. The results, including determined levels of SN-38 and SN-38G in plasma are presented in Tables 2 and 3 below; and the calculated values of area under the curve (AUC) for SN-38 and SN-38G are presented in the Table 4.

TABLE 2
Plasma levels of SN38 and SN-38G upon dosing of a composition
comprising SN-38, SBECD, and heptakis(6-amino-6-deoxy)-
beta-cyclodextrin (H6A); SN-38 dose 0.65 mg/kg

Plasma levels [ng/mL]

| | | | |
|---|---|---|---|
| SN-38 | SN-38G | SN-38 | SN-38G |
| [ng/mL] | [ng/mL] | [ng/mL] | [ng/mL] |

| | | |
|---|---|---|
| | Dosing SN-38 | Dosing SN-38 |
| Time | 0.65 mg/kg in 40% | 0.65 mg/kg in 40% |
| [h] | SBECD, 1% H6A | SBECD, 2% H6A |

| | | | | |
|---|---|---|---|---|
| 0.17 | 154.3 | 344.5 | 132.9 | 297.2 |
| 0.5 | 60.4 | 306.0 | 58.0 | 215.2 |
| 0.75 | 44.5 | 255.5 | 35.5 | 165.6 |
| 1 | 28.8 | 187.0 | 21.9 | 140.9 |
| 2 | 10.1 | 75.7 | 10.3 | 48.8 |
| 4 | 5.5 | 19.8 | 5.9 | 17.4 |
| 6 | 1.7 | 9.8 | 4.9 | 3.8 |

TABLE 3
Plasma levels of SN38 and SN-38G upon dosing of a composition
comprising SN-38, SBECD, and heptakis(6-amino-6-deoxy)-
beta-cyclodextrin (H6A); SN-38 dose 2 mg/kg

Plasma levels [ng/mL]

| | | | |
|---|---|---|---|
| SN-38 | SN-38G | SN-38 | SN-38G |
| [ng/mL] | [ng/mL] | [ng/mL] | [ng/mL] |

| | | |
|---|---|---|
| | Dosing SN-38 | Dosing SN-38 |
| Time | 2 mg/kg in 40% | 2 mg/kg in 40% |
| [h] | SBECD, 1% H6A | SBECD, 2% H6A |

| | | | | |
|---|---|---|---|---|
| 0.17 | 950.7 | 2324.6 | 1060.2 | 1761.9 |
| 0.5 | 383.1 | 1463.5 | 440.6 | 1209.9 |
| 0.75 | 240.0 | 958.0 | 233.0 | 841.0 |
| 1 | 147.0 | 711.3 | 150.1 | 722.3 |
| 3 | 10.0 | 94.0 | 11.0 | 145.0 |
| 6 | 6.5 | 38.6 | 5.8 | 34.3 |

TABLE 4
Areas under the curve (AUC)

| | SN-38 AUC | SN-38G AUC | Ratio AUC |
|---|---|---|---|
| Dosing | [ng*h/mL] | [ng*h/mL] | SN-38/SN-38G |
| SN-38 0.65 mg/kg in | 122 | 538 | 0.23 |
| 40% SBECD, 1% H6A | | | |
| SN-38 0.65 mg/kg in | 116 | 403 | 0.29 |
| 40% SBECD, 2% H6A | | | |
| SN-38 2 mg/kg in | 604 | 2805 | 0.22 |
| 40% SBECD, 1% H6A | | | |
| SN-38 2 mg/kg in | 634 | 2175 | 0.29 |
| 40% SBECD, 2% H6A | | | |

These results demonstrate that the compositions of the present invention provide the pharmaceutical agent (SN-38) with systemic bioavailability as demonstrated by the formation of the main metabolite of the compound, SN-38G.

### Example 13

Pharmacokinetics of SN-38 and SN-38 glucuronide (SN-38G) upon dosing of a composition comprising SN-38, SBECD, and hexakis(6-amino-6-deoxy)-alpha-cyclodextrin (“AH6A”) or octakis(6-amino-6-deoxy)-gamma-cyclodextrin (“06A”) intravenously to rats.

Female Sprague-Dawley rats, 4 animals per group received i.v. injections of SN-38 (0.65 mg/kg) in 40% SBECD with either 2% AH6A, or 1% 06A, or 2% O6A. Blood plasma samples were collected from each group of animals, extracted using liquid extraction with cold methanol/acetonitrile mixture (1:1 v/v), and analyzed using a HPLC method with fluorescent detection. The results, including the determined levels of SN-38 and SN-38G in plasma and calculated values of area under the curve (AUC) for SN-38 and SN-38G are presented in Tables 5 and 6 below.

TABLE 5
Plasma levels of SN38 and SN-38G upon dosing of a composition
comprising SN-38, SBECD, and hexakis(6-amino-6-deoxy)-alpha-
cyclodextrin (AH6A) or octakis(6-amino-6-deoxy)-gamma-
cyclodextrin (O6A)

| | Plasma levels [ng/mL] | | | | | |
|---|---|---|---|---|---|---|
| | SN-38 | SN-38G | SN-38 | SN-38G | SN-38 | SN-38G |
| | [ng/mL] | [ng/mL] | [ng/mL] | [ng/mL] | [ng/mL] | [ng/mL] |
| | Dosing SN-38 | | Dosing SN-38 | | Dosing SN-38 | |
| | 0.65 mg/kg in | | 0.65 mg/kg in | | 0.65 mg/kg in | |
| Time | 40% SBECD, | | 40% SBECD, | | 40% SBECD, | |
| [h] | 2% AH6A | | 1% O6A | | 2% O6A | |
| 0.17 | 186.6 | 139.4 | 222.6 | 289.0 | 198.6 | 283.2 |
| 0.5 | 51.9 | 108.1 | 95.6 | 259.7 | 85.2 | 226.8 |
| 0.75 | 26.8 | 86.9 | 50.7 | 217.0 | 63.8 | 195.4 |
| 1 | 14.4 | 50.4 | 30.3 | 154.6 | 49.4 | 152.3 |
| 2 | 6.0 | 15.6 | 11.5 | 59.7 | 11.5 | 55.4 |
| 4 | 6.0 | 7.3 | 7.0 | 17.1 | 5.5 | 13.2 |
| 6 | | | 1.7 | | | 1.6 |

TABLE 6
Areas under the Curve (AUC)

| | SN-38 AUC | SN-38G AUC | Ratio AUC |
|---|---|---|---|
| Dosing | [ng*h/mL] | [ng*h/mL] | SN-38/SN-38G |
| SN-38 0.65 mg/kg in | 111 | 169 | 0.66 |
| 40% SBECD, 2% AH6A | | | |
| SN-38 0.65 mg/kg in | 166 | 447 | 0.37 |
| 40% SBECD, 1% O6A | | | |
| SN-38 0.65 mg/kg in | 166 | 416 | 0.40 |
| 40% SBECD, 2% O6A | | | |

The above results demonstrate that the compositions of the present invention provide the pharmaceutical agent (SN-38) with systemic bioavailability as demonstrated by the formation of the main metabolite of the compound, SN-38G.

### Example 14

Pharmacokinetics of SN-38 and SN-38 glucuronide (SN-38G) upon dosing with a composition comprising SN-38, SBECD, and heptakis(6-guanidino-6-deoxy)-beta-cyclodextrin (H6G) or octakis(6-guanidino-6-deoxy)-gamma-cyclodextrin (O6G) intravenously to rats.

Female Sprague-Dawley rats, 4 animals per group received i.v. injections of SN-38 (0.65 mg/kg) in 40% SBECD with either 2% H6G, or 2% O6G. Blood plasma samples were collected from each group of animals, extracted using liquid extraction with cold methanol/acetonitrile mixture (1:1 v/v), and analyzed using a HPLC method with fluorescence detection. The results, including determined levels of SN-38 and SN-38G in plasma and calculated values of area under the curve (AUC) for SN-38 and SN-38G are presented in Tables 7 and 8 below.

TABLE 7
Plasma levels of SN38 and SN-38G upon dosing of composition
comprising SN-38, SBECD, and heptakis(6-guanidino-
6-deoxy)-beta-cyclodextrin (H6G) or octakis(6-guanidino-
6-deoxy)-gamma-cyclodextrin (O6G)

Plasma levels [ng/mL]

| | | | |
|---|---|---|---|
| SN-38 | SN-38G | SN-38 | SN-38G |
| [ng/mL] | [ng/mL] | [ng/mL] | [ng/mL] |

| | | |
|---|---|---|
| | Dosing SN-38 | Dosing SN-38 |
| Time | 0.65 mg/kg in 40% | 0.65 mg/kg in 40% |
| [h] | SBECD, 2% H6G | SBECD, 2% O6G |

| | | | | |
|---|---|---|---|---|
| 0.17 | 188.3 | 145.3 | 209.8 | 211.1 |
| 0.5 | 87.1 | 138.1 | 105.3 | 194.3 |
| 0.75 | 74.5 | 124.2 | 72.7 | 183.7 |
| 1 | 50.3 | 110.8 | 63.8 | 175.5 |
| 2 | 34.4 | 82.3 | 15.0 | 94.8 |
| 4 | 12.1 | 42.1 | 7.0 | 24.6 |
| 6 | 8.1 | 25.8 | 4.9 | 10.0 |

TABLE 8
Areas under the Curve

| | SN-38 AUC | SN-38G AUC | Ratio AUC |
|---|---|---|---|
| Dosing | [ng*h/mL] | [ng*h/mL] | SN-38/SN-38G |
| SN-38 0.65 mg/kg in | 222 | 443 | 0.53 |
| 40% SBECD, 2% H6G | | | |
| SN-38 0.65 mg/kg in | 200 | 484 | 0.41 |
| 40% SBECD, 2% O6G | | | |

These results demonstrate that the compositions of the present invention provide the pharmaceutical agent (SN-38) with systemic bioavailability as demonstrated by the formation of the main metabolite of the compound, SN-38G.

### Example 15

### Effect of SN-38 Compositions on the Growth of Subcutaneous Solid Tumor of Human Colon Carcinoma LoVo DX Cells in Balb/c Mice

Lovo Dx cells (2.5×10⁶ cells per an injection) in culture medium with 30% Matrigel were subcutaneously inoculated at 2 sides of the flank (in the mid-flank) of each of 22 Balb/c mice. 20 days after inoculation the animals were randomly divided into 3 groups: control (8 mice) and treated (two groups of 7 animals). On day 21, 24, 27 and 30 after inoculation (days 1, 4, 7, and 10 of treatment), twice daily, at 10 am and at 4 pm, the animals received intraperitoneal injections. Control animals received each time injection of 0.49 mL of 0.9% saline. One group of treated animals received each time injection of SN-38 solution in 40% SBECD (w/w) and 1% H6A, 4.5 mg/kg of SN-38. Another group of treated animals received each time injection of SN-38 solution in 40% SBECD (w/w) and 1% O6A, 4.5 mg/kg of SN-38. Tumor size and body weight of each animal was monitored during treatment. The results represented as average tumor volume estimated from measurements of tumor diameters (V=0.5*D1*D2*D2, where D1 and D2 are longer and shorter diameter of the tumor) are presented in Table 9 below (the standard error of mean is in parenthesis).

TABLE 9
Tumor volume and its change upon treatment

| | | | Group treated | | Group treated | |
|---|---|---|---|---|---|---|
| | Control group | | with SN-38 in | | withSN-38 in | |
| | treated with | | 20% SBECD, | | 20% SBECD, | |
| | 0.9% NaCl | | 0.5% H6A | | 0.5% O6A | |
| Day of | Tumor | Tumor | Tumor | Tumor | Tumor | Tumor |
| treat- | volume | volume | volume | volume | volume | volume |
| ment | [cm³] | increase* | [cm³] | increase* | [cm³] | increase* |
| 0 | 0.119 | 100 | 0.117 | 100 | 0.118 | 100 |
| | (0.009) | | (0.011) | | (0.011) | |
| 4 | 0.133 | 110 (4) | 0.114 | 103 (9) | 0.129 | 112 (6) |
| | (0.013) | | (0.009) | | (0.010) | |
| 6 | 0.151 | 128 (6) | 0.122 | 111 (9) | 0.137 | 126 (7) |
| | (0.013) | | (0.009) | | (0.012) | |
| 8 | 0.179 | 151 (8) | 0.130 | 118 (10) | 0.141 | 133 (10) |
| | (0.016) | | (0.010) | | (0.011) | |
| 11 | 0.239 | 202 (12) | 0.155 | 138 (11) | 0.164 | 152 (11) |
| | (0.021) | | (0.013) | | (0.016) | |
| 13 | 0.273 | 233 (17) | 0.164 | 148 (14) | 0.176 | 163 (11) |
| | (0.026) | | (0.013) | | (0.016) | |
| 15 | 0.305 | 261 (21) | 0.192 | 177 (20) | 0.190 | 175 (13) |
| | (0.03) | | (0.017) | | (0.017) | |
| 18 | 0.425 | 364 (37) | 0.244 | 232 (35) | 0.218 | 201 (10) |
| | (0.051) | | (0.026) | | (0.019) | |
| 20 | 0.584 | 504 (52) | 0.323 | 306 (47) | 0.276 | 258 (21) |
| | (0.063) | | (0.037) | | (0.027) | |
| 22 | 0.804 | 686 (69) | 0.414 | 396 (68) | 0.327 | 305 (24) |
| | (0.091) | | (0.053) | | (0.034) | |
| p** | | | 0.011 | | 0.024 | |

*Percent relative to day 0 of treatment
**p - two-tailed paired t-test vs. control

The above results demonstrate that the compositions of the present invention provide the pharmaceutical agent (SN-38) with systemic bioavailability as demonstrated by its anti-cancer activity.

### Example 16

### Effect of SN-38 Composition on the Growth of Subcutaneous Solid Tumor of Human Breast Carcinoma MDA-MB-231 Cells in Balb/c Mice

MDA-MB-231 cells (5×10⁵ cells per an injection) in culture medium with 30% Matrigel were subcutaneously inoculated at 2 sides of the flank (in the mid-flank) of each of 20 Balb/c mice. 26 days after inoculation the animals were be randomly divided into 4 groups of 5 animals: control and three treated groups. On day 27, 30, 33, 36 and 40 after inoculation (days 1, 4, 7, 10 and 14 of treatment), twice daily, at 10 am and at 4 pm, the animals received intraperitoneal injections. Control animals received each time injection of 0.49 mL of 0.9% saline. The groups of treated animals received each time injection of 4.5 mg/kg SN-38 solution in 20% SBECD (w/w) and respectively: 0.5% H6A, 0.5% H6G and 0.5% O6G. Tumor sizes and body weight of each animal was monitored during treatment. The results represented as average tumor volume estimated from measurements of tumor diameters (V=0.5*D1*D2*D2, where D1 and D2 are longer and shorter diameter of the tumor) are presented in Table 10 below (the standard error of mean is in parenthesis).

TABLE 10
Tumor volume and its change upon treatment

| | | | Group treated | | Group treated | | Group treated | |
|---|---|---|---|---|---|---|---|---|
| | Control group | | with SN-38 in | | with SN-38 in | | with SN-38 in | |
| | treated with | | 20% SBECD, | | 20%SBECD, | | 20% SBECD, | |
| | 0.9% NaCl | | 0.5% H6A | | 0.5% H6G | | 0.5% O6A | |
| | Tumor | *Tumor | Tumor | *Tumor | Tumor | *Tumor | Tumor | *Tumor |
| Day of | volume | volume | volume | volume | volume | volume | volume | volume |
| treatment | [cm³] | increase | [cm³] | increase | [cm³] | increase | [cm³] | increase |
| 0 | 0.16 | 100 | 0.17 | 100 | 0.17 | 100 | 0.17 | 100 |
| | (0.03) | | (0.03) | | (0.03) | | (0.04) | |
| 2 | 0.24 | 149 (6) | 0.21 | 124 (6) | 0.23 | 134 (7) | 0.24 | 139 (9) |
| | (0.05) | | (0.05) | | (0.05) | | (0.06) | |
| 4 | 0.30 | 188 (10) | 0.25 | 146 (9) | 0.30 | 154 (7) | 0.29 | 174 (16) |
| | (0.05) | | (0.05) | | (0.07) | | (0.08) | |
| 7 | 0.41 | 256 (14) | 0.34 | 190 (16) | 0.38 | 189 (6) | 0.38 | 228 (21) |
| | (0.09) | | (0.08) | | (0.09) | | (0.10) | |
| 9 | 0.48 | 291 (20) | 0.39 | 230 (15) | 0.42 | 220 (9) | 0.45 | 258 (32) |
| | (0.12) | | (0.08) | | (0.09) | | (0.15) | |
| 11 | 0.61 | 361 (27) | 0.47 | 270 (16) | 0.49 | 259 (13) | 0.52 | 296 (28) |
| | (0.15) | | (0.11) | | (0.10) | | (0.16) | |
| 14 | 0.84 | 520 (48) | 0.72 | 354 (29) | 0.63 | 336 (19) | 0.67 | 380 (37) |
| | (0.19) | | (0.16) | | (0.14) | | (0.18) | |
| 16 | 1.09 | 673 (59) | 0.96 | 473 (41) | 0.86 | 444 (29) | 0.86 | 511 (38) |
| | (0.24) | | (0.22) | | (0.20) | | (0.21) | |
| 18 | 1.35 | 849 (66) | 1.14 | 565 (44) | 1.05 | 552 (36) | 0.97 | 580 (48) |
| | (0.28) | | (0.25) | | (0.25) | | (0.24) | |
| p** | | | | 0.010 | | 0.012 | | 0.030 |

*Percent relative to day 0 of treatment
**p - two-tailed paired t-test vs. control

The results above demonstrate that the compositions of the present invention provide the pharmaceutical agent (SN-38) with systemic bioavailability as demonstrated by its anti-cancer activity.

### Example 17

### Effect of Doses of SN-38 Composition on Formation of Lung Metastasis in C57BL/6 Mice After i.v. Implantation of 3LL Cell

3LL cells (2×10⁵ cells per an injection) in culture medium were intravenously inoculated via the tail vein to C57BL/6 mice. The animals were randomly divided into 3 groups, and treated with intraperitoneal (i.p.) injections once daily on days 1, 4, 7, and 10 after inoculation. The control group received i.p. injections of 0.9% NaCl. The two groups of treated animals received i.p. injections of SN-38 in composition with 20% SBECD and 0.5% H6A. One group received a dosage of 5 mg/kg in each injection. The other group received dosages of 10 mg/kg, 5 mg/kg, 5 mg/kg and 10 mg/kg on days 1, 4, 7 and 10 respectively. On day 14 the animals were sacrificed, lungs were harvested and metastasis spots in lungs were counted. Table 11 below presents the average number of metastasis observed.

TABLE 11

| Treatment (days | Metastasis count | | | Infibition of |
|---|---|---|---|---|
| 1, 4, 7, and 10) | Mean | SEM* | p** | metastasis |
| Control 0.9% NaCl | 78 | 9 | | |
| SN-38 5 mg/kg in | 44 | 10 | 0.017 | 44% |
| 20% SBECD, 0.5% H6A | | | | |
| SN-38 doses 10, 5, 5, | 7.6 | 3 | 0.0003 | 90% |
| and 10 mg/kg in 20% | | | | |
| SBECD, 0.5% H6A | | | | |

*SEM—standard error of mean
**p—Two-tailed Mann Whitney test (non parametric) vs. control

The results above demonstrate that the compositions of the present invention provide the pharmaceutical agent (SN-38) with systemic bioavailability as demonstrated by its anti-cancer activity.

### Example 18

### Effect of SN-38 Compositions on the Formation of Lung Metastasis in C57BL/6 Mice After i.v. Implantation of 3LL Cells

3LL cells (2×10⁵ cells per an injection) in culture medium were intravenously inoculated via the tail vein of to C57BL/6 mice. The animals were randomly divided into 5 groups and, on day 1 after inoculation at 9 am and 4 pm, were treated with intraperitoneal (i.p.) injections. The control group received i.p. injections of 0.9% NaCl. The groups of treated animals received total dose of 10 mg/kg of SN-38 in the following compositions: 40% SBECD+1% H6A, 20% SBECD+0.5% H6A, 10% SBECD+0.25% H6A, 8.5% SBECD+0.213% H6A. On day 14 the animals were sacrificed, their lungs were harvested and the number of metastasis spots in the lungs counted. The average number of metastasis observed is present in Table 12.

TABLE 12

| | Metastasis count | | | Infibition of |
|---|---|---|---|---|
| Treatment | Mean | SEM* | p** | metastasis |
| Control 0.9% NaCl | 58 | 8 | | |
| SN-38 10 mg/kg in | 9 | 2 | <0.0001 | 85% |
| 40% SBECD, 1% H6A | | | | |
| SN-38 10 mg/kg in | 11 | 3 | <0.0001 | 81% |
| 20% SBECD, 0.5% H6A | | | | |
| SN-38 10 mg/kg in | 7 | 1 | <0.0001 | 88% |
| 10% SBECD, 0.25% H6A | | | | |
| SN-38 10 mg/kg in | 9 | 2 | <0.0001 | 84% |
| 8.5% SBECD, 0.21% H6A | | | | |

*SEM—standard error of mean
**p—Two-tailed Mann Whitney test (non parametric) vs. control

The results above demonstrate that the compositions of the present invention provide the pharmaceutical agent (SN-38) with systemic bioavailability as demonstrated by its anti-cancer activity.

### Example 19

### Effect of SN-38 Composition on Formation of Lung Metastasis in C57BL/6 Mice After i.v. Implantation of 3LL Cells

3LL cells (2×10⁵ cells per an injection) in culture medium were intravenously inoculated via the tail vein to C57BL/6 mice. The animals were randomly divided into 5 groups, and treated twice daily on the day 1 after inoculation. Three control groups received i.p. injections of 0.9% NaCl, 20% SBECD+1% H6A, and 20% SBECD+1% H6G, respectively. The two groups of treated animals received total dose 5 mg/kg of SN-38 in the following compositions: 20% SBECD+1% H6A and 20% SBECD+1% H6G, respectively. On day 14 the animals were sacrificed, their lungs harvested and the number of metastasis spots in the lungs counted. Table 13 below presents the average number of metastasis spots observed.

TABLE 13

| | Metastasis count | | | Infibition of |
|---|---|---|---|---|
| Treatment | Mean | SEM* | p** | metastasis |
| Control 0.9% NaCl | 34 | 5 | | |
| Control 20% SBECD, | 36 | 7 | 0.73 | — |
| 1% H6A | | | | |
| Control 20% SBECD, | 38 | 10 | 0.66 | — |
| 1% H6G | | | | |
| SN-38 5 mg/kg in | 3 | 0.5 | 0.0004 | 92% |
| 20% SBECD, 1% H6A | | | | |
| SN-38 5 mg/kg in | 2 | 0.5 | 0.0008 | 95% |
| 20% SBECD, 1% H6G | | | | |

*SEM—standard error of mean
**p—Two-tailed Mann Whitney test (non parametric) vs. control

The results above demonstrate that the compositions of the present invention provide the pharmaceutical agent (SN-38) with systemic bioavailability as demonstrated by its anti-cancer activity.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A composition comprising:
(a) an active ingredient;
(b) a charged cyclopolysaccharide comprising at least one charged group; and
(c) a stabilizing agent comprising at least one charged group having a charge opposite to that of the cyclopolysaccharide, with the proviso that the active ingredient is other than bendamustine.

2. The composition of claim 1 wherein the cyclopolysaccharide is a beta-cyclodextrin.

3. The composition of claim 1 wherein the charged group on the cyclopolysaccharide is an anionic group.

4. The composition of claim 3 wherein the anionic group is selected from the group consisting of is selected from the group consisting of sulphate, sulphonyl, and carbonyl groups.

5. The composition of claim 3 wherein the anionic cyclopolysaccharide is selected from the group consisting of sulfobutyl ether beta-cyclodextrin, carboxymethylated-beta-cyclodextrin, O-phosphated-beta-cyclodextrin, succinyl-(2-hydroxy)propyl-beta-cyclodextrin and sulfopropylated-beta-cyclodextrin, or their pharmaceutically acceptable salts.

6. The composition of claim 3 wherein the stabilizing agent is selected from the group consisting of primary amines, secondary amines, tertiary amines, quarternary ammonium compounds, polyamines, pegylated polyamines, cationic polypeptides, cationic polysaccharides, polycationic polymers and cationic cyclopolysaccharide compounds, or their pharmaceutically acceptable salts.

7. The composition of claim 3 wherein the stabilizing agent is a polypeptide comprising from about 5 to about 50 amino acids, wherein at least about 50% of such amino acids contain a positively chargeable groups.

8. The composition of claim 7 wherein such polypeptide comprises between about 6 and about 20 amino acids.

9. The composition of claim 8 wherein the polypeptide comprises at least one block sequence of 4 arginines.

10. The composition of claim 3 wherein the stabilizing agent is polyarginine.

11. The composition of claim 3 wherein the stabilizing agent is low molecular weight protamine.

12. The composition of claim 3 wherein the stabilizing group is a charged group on the cyclopolysaccharide is a cationic cyclopolysaccharide.

13. The composition of claim 12 wherein the cationic cyclopolysaccharide is selected from the group consisting of hexakis(6-amino-6-deoxy)alpha-cyclodextrin, heptakis(6-amino-6-deoxy)beta-cyclodextrin, octakis(6-amino-6-deoxy)gamma-cyclodextrin, heptakis(6-guanidino-6-deoxy)beta-cyclodextrin, octakis(6-guanidino-6-deoxy)-gamma-cyclodextrin, 2-hydroxy-N,N,N-trimethylpropanammonium-cyclodextrin and 6-deoxy-6-(3-hydroxy)propylamino beta-cyclodextrin, or their pharmaceutically acceptable salts.

14. The composition of claim 1 wherein the active ingredient is SN-38.

15. The composition of claim 1 wherein the charged polysaccharide (b) is a cationic polysaccharide.

16. The composition of claim 15 wherein the cationic cyclopolysaccharide is selected from the group consisting of hexakis(6-amino-6-deoxy)alpha-cyclodextrin, heptakis(6-amino-6-deoxy)beta-cyclodextrin, octakis(6-amino-6-deoxy)gamma-cyclodextrin, heptakis(6-guanidino-6-deoxy)beta-cyclodextrin, octakis(6-guanidino-6-deoxy)-gamma-cyclodextrin, 2-hydroxy-N,N,N-trimethylpropanammonium-cyclodextrin and 6-deoxy-6-(3-hydroxy)propylamino beta-cyclodextrin, or their pharmaceutically acceptable salts.

17. The composition of claim 15 wherein the stabilizing agent is selected from the group consisting of anionic surfactants, anionic polysaccharides, polyanionic polymers and anionic cyclopolysacharides.

18. The composition of claim 15 wherein the stabilizing agent is an anionic cyclodextrin.

19. The composition of claim 18 wherein the anionic cyclodextrin is selected from the group consisting of sulfobutyl ether beta-cyclodextrin, carboxymethylated-beta-cyclodextrin, O-phosphated-beta-cyclodextrin, succinyl-(2-hydroxy)propyl-beta-cyclodextrin and sulfopropylated-beta-cyclodextrin, or pharmaceutically acceptable salts thereof.
